(19) United States
(12) Patent Application Publication
(10) Pub. No.: US 2021/0236547 A1
(43) Pub. Date: Aug. 5, 2021

(54) RAPID PRODUCTION, EXPANSION, AND INCREASED PURITY OF CAR-T CELLS USING BEADS WITH PROTEIN L

(71) Applicant: H. LEE MOFFITT CANCER CENTER AND RESEARCH INSTITUTE, INC., Tampa, FL (US)

(72) Inventor: Marco Davila, Tampa, FL (US)

(21) Appl. No.: 16/972,223

(22) PCT Filed: Jun. 5, 2019
(86) PCT No.: PCT/US2019/035547

§ 371 Date: Dec. 4, 2020

Related U.S. Application Data
(60) Provisional application No. 62/681,029, filed on Jun. 5, 2018.

(51) Int. Cl. A61K 35/17; C07K 14/195; C07K 14/725; C12N 5/0783; A61P 35/00

(52) U.S. Cl. CPC A61K 35/17; C07K 14/195; A61P 35/00; C12N 5/0637; C07K 14/7051

(57) ABSTRACT

Disclosed herein are methods of expanding immune cells for immunotherapy and/or increasing the purity of a population of CAR T cells using an immune and/or magnetic bead; wherein the beads comprises Protein L on its surface. The disclosed beads can also comprise antibodies that bind molecules of the T cell inhibitory pathway. For example, anti-CD3 scFv on the surface of the beads can bind and activate T cells, while anti-CD28 scFv and 4-1BBL on the surface of the beads can provide dual co-stimulation for the T cells resulting in decreased levels of the markers CD25, TIM3, LAG3, and PD1. For example, blocking PD1/PDL1 ligation can limit suppression that is mediated by the tumor microenvironment. This is a less costly and more efficient alternative to peripheral blood mononuclear cells (PBMCs) and cytokine treatments that result in better quality T cell for adoptive transfer back into patients.

### BACKGROUND

Adoptive cell therapy (ACT) using chimeric antigen receptor (CAR) T cells, tumor infiltrating lymphocytes (TIL), and/or marrow-infiltrating lymphocytes (MIL) can lead to positive, objective, and durable responses in cancer patients. However, this therapy can involve sophisticated cell processing techniques and equipment. These procedures have introduced technical, regulatory, and logistic challenges to the successful use of CAR T cells, MIL, TIL as a biological therapy. Accordingly, there is a need in the art for improved methods for growing CAR T cells, MIL, and/or TIL for use in adoptive cell therapy.

### SUMMARY

Disclosed herein are methods of expanding immune cells for immunotherapy using immuno and/or multiplexed beads having on their surface Protein L and/or antibodies (including, but not limited to antibody fragments, such as, for example, F(ab′)2, Fab′, Fab, and/or scFv) and/or ligands that bind molecules of both the T cell activation pathway and T cell co-stimulation pathway. The disclosed beads can also comprise antibodies that bind molecules of the T cell inhibitory pathway. For example, anti-CD3 scFv on the surface of the beads can bind and activate T cells, while anti-CD28 scFv and 4-1BBL on the surface of the beads can provide dual co-stimulation for the T cells resulting in decreased levels of the markers CD25, TIM3, LAGS, and PD1. This is a less costly and more efficient alternative to peripheral blood mononuclear cells (PBMCs) and cytokine treatments that result in better quality T cell for adoptive transfer back into patients.

In some embodiments, the disclosed beads comprise an antibody (e.g. anti-PD1 or PDL1) that interferes with suppression of T cells, e.g. by ligation of PD1 with PDL1. This suppression is a normal physiologic immune response meant to prevent over-activation of T cells. However, cancer cells have co-opted this suppression pathway as a means to evade immune recognition and tumor killing. This system is a less costly, more efficient and more rapid alternative to peripheral blood mononuclear cells (PBMCs) and cytokine treatments. The system is less costly because a renewable resource replaces the need for cytokines, antibodies for activation, and PBMC feeders. The faster production time is also clinically meaningful considering that patients have to wait a few months for production of their cells, which can be a difficult task for patients with metastatic cancer. Also, extended culture often produce terminally differentiated T cells that have limited function and persistence when adoptively transferred back into patients. The shorter culture time therefore may allow us to infuse a T cell product that is more physiologic and tumor-reactive. In one aspect, the T cells are added to culture containers pre-treated with RetroNectin® and incubated with viral particles for gene transfer. The cells, beads and virus are incubated and the gene-modified cells are expanded.

Also disclosed herein are methods of increasing the purity of CAR T cells in a population of immune cells, comprising providing an immuno and/or multiplexed beads, wherein the beads comprise Protein L on its surface; and incubating the immune cell population with the beads for at least 8, 9, 10, 11, 12, 13, 14, 15, 16, 17, 18, 19, 20, 21, 22, 23, 24, 26, 28, 30, 32, 34, 36, or 48 hours; wherein the beads induces the CAR T cells to proliferate

The details of one or more embodiments of the invention are set forth in the accompanying drawings and the description below. Other features, objects, and advantages of the invention will be apparent from the description and drawings, and from the claims.

### DETAILED DESCRIPTION

Disclosed herein are methods for expanding an immune cell for use in immune therapy. The disclosed methods comprise providing an immuno and/or multiplexed beads, wherein the beads comprise Protein L on its surface. Protein L is a bacterial protein from Peptostreptococcus magnus that binds to the kappa light chain of antibodies and is therefore its binding is not limited to full antibodies, but can also bind antibody fragments such as scFv and Fab fragments. Of note, the ability to bind the kappa light chain allows for the bead to bind the antigen-binding domain of CAR T cells. Because Protein L is not native to mammals, Protein L can be used as a target for antibody based detection or purification methods. For example, an antibody specific for Protein L can be used to detect or isolate CAR T cells that are bound by Protein L. Accordingly, in one aspect, disclosed herein are methods for expanding an immune cell for use in immune therapy comprising providing immuno and/or multiplexed beads; wherein the beads comprise Protein L, and comprises Protein L; and contacting the isolated immune cell with an effective amount of the beads to expand the immune cell in an amount effective for immunotherapy.

As noted above, Protein L is not a native protein to mammalian cells and thus its ability to bind to kappa light chains can be used as a marker for cells bound to Protein L, such as, for example CAR T cells. In one aspect, the binding of the Protein L marker can be used to identify, expand, enrich, and/or purify an immune cell population for cells bound by Protein L (for example, CAR T cells). Protein L detection can be achieved via antibodies that specifically bind to Protein L. Therefore, any purification and detection methods employing antibodies (including tagged antibodies for use in flow cytometry, multiplexed bead arrays, immunobead capture assays (such as, magnetic bead capture), ELISAs, ELlspots, and florescence acquired cell sorting (FACS)) can be used to identify, detect, enrich, and/or purify cell bound to protein L. Thus, in one aspect, disclosed herein are method for increasing the purity of immune cells (such as, for example CAR T cells, TILs, MILs, NK cells, NK-T cell, cytokine-induced memory NK cells, a cytokine-induced killer (CIK) cells, and/or γδ T cells) in a mixed population of immune cells (such as, for example, a population of immune cells comprising CD4 T cells, CD8 T cells, NK cells, CIK cells, γδ T cells, macrophage, and/or B cells), comprising a) providing an immuno and/or multiplexed bead comprising Protein L; and b) incubating the immune cell population with the beads; wherein the beads induces only the CAR T cells to proliferate. In one aspect, the method can further comprise separating the CAR T cells bound to Protein L via FACS or immunobead capture (such as, magnetic bead capture or microbeads).

It is understood and herein contemplated that by incubating the beads in an immune cell population, the beads will selectively stimulate and induce proliferation of the cells to which they bind (for example, CAR T cells). As the incubation time increases so too does the purity of the beads bound cell population. Thus, in one aspect, disclosed herein are methods of increasing the purity of immune cells (such as, for example, CAR T cells), wherein the immune cells are incubated with the beads for at least for at least 8, 9, 10, 11, 12, 13, 14, 15, 16, 17, 18, 19, 20, 21, 22, 23, 24, 26, 28, 30, 32, 34, 36, 48, 60 hours, 3, 4, 5, 6, 7, 8, 9, 10, 11, 12, 13, 14, 15, 16, 17, 18, 19, 20, 21, 28, or 30 days.

It is understood and herein contemplated that the immune cell used in the disclosed methods can be isolated from a subject receiving the immune therapy (an autologous donor source), from a type match donor source (i.e., syngeneic), from a non-type matched donor source of the same species (i.e, an allogeneic source), a donor of a different species (xenogeneic source), or cell line. In one aspect, the immune cell can be a chimeric antigen receptor (CAR) T cell, tumor infiltrating lymphocyte (TIL), or marrow-infiltrating lymphocyte (MIL). In other aspects, the immune cell can comprise a natural killer (NK) cell, an NK-T cell, a cytokine-induced memory NK cell, a cytokine-induced killer (CIK) cell, or γδ T cell.

In one aspect, it is understood and herein contemplated that the beads comprising Protein L can be further engineered to enhance the beads ability to bind T cells and stimulate proliferation. For example, an antibody or antibody fragment (such as, for example, scFv) that binds to the T cell receptor (CD3) or that binds to a co-stimulatory molecule on T cells (for example, CD28 or 4-1BB). Where the immune cell to be expanded is an NK cell, the co-stimulator molecule ca be CD80 or CD86. Accordingly, in one aspect disclosed herein are methods of methods for expanding an immune cell for use in immune therapy or methods for increasing the purity of CAR T cells said methods comprising providing an beads; wherein the beads comprise Protein L on their surface and wherein the beads further comprises one or more scFv or ligands that bind a co-stimulatory molecule on T-cells (such as, for example, CD28 and/or 4-1BB), one or more scFv that selectively bind CD3, and/or a combination thereof on the bead surface.

The term “antibody” refers to natural or synthetic antibodies that selectively bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that selectively bind the target antigen.

As used herein, the term “antibody or fragments thereof” encompasses chimeric antibodies and hybrid antibodies, with dual or multiple antigen or epitope specificities, and fragments, such as F(ab′)2, Fab′, Fab, scFv, and the like, including hybrid fragments. Thus, fragments of the antibodies that retain the ability to bind their specific antigens are provided. For example, fragments of antibodies which maintain CD3, CD28, CD137, PD1, CTLA4, LAGS, TIM3, BTLA, CD160, 2B4, A2aR, and KIR binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans Therefore, the use of human or humanized antibodies in the methods serves to lessen the chance that an antibody administered to a human will evoke an undesirable immune response.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

In one aspect, the beads comprising Protein L disclosed herein can comprise surface bound antibodies or antibody fragments that bind molecules of the T cell inhibitory pathway. In some embodiments, the disclosed beads comprise an antibody or antibody fragment (for example, an scFv) that interferes with suppression of T cells, e.g. by ligation of PD1 with PDL1 such as, for example, use of an anti-PD1 or PDL1 antibody or antibody fragment. This suppression is a normal physiologic immune response meant to prevent over-activation of T cells. However, cancer cells have co-opted this suppression pathway as a means to evade immune recognition and tumor killing. This system is a less costly, more efficient and more rapid alternative to peripheral blood mononuclear cells (PBMCs) and cytokine treatments. The system is less costly because a renewable resource replaces the need for cytokines, antibodies for activation, and PBMC feeders. The faster production time is also clinically meaningful considering that patients have to wait a few months for production of their cells, which can be a difficult task for patients with metastatic cancer. Also, extended culture often produce terminally differentiated T cells that have limited function and persistence when adoptively transferred back into patients. The shorter culture time therefore may allow us to infuse a T cell product that is more physiologic and tumor-reactive. In one aspect, other immune cell inhibitory molecule comprises CTLA4, LAG3, TIM3, BTLA, CD160, 2B4, A2aR, PD-1, ICOS, CD25, TIM3, LAG3, PD1, CD40, CD137, OX40, CD2, LFA-1, CD28, CD154, BTLA, CD160, TIM 1, TIM 4, KIR, any glucocorticoid-induced tumor necrosis factor-related receptor (GITR), and/or any combination thereof. Thus, in one aspect, disclosed herein are methods for expanding an immune cell isolated from a subject for autologous immune therapy and/or methods for increasing the purity of CAR T cells said methods comprising a) providing an immuno and/or multiplexed bead comprising Protein L and comprises Protein L on its surface; wherein the cell comprises one or more single chain variable fragment (scFv) antibodies that bind a T cell inhibitory molecule, or a combination thereof; and b) contacting the isolated immune cell with an effective amount of the beads to expand the immune cell in an amount effective for immunotherapy. In one aspect, the beads can further comprise on their surface: one or more scFv that selectively bind an immune cell selective receptor (such as, for example CD3) and one or more scFv or ligands that bind a co-stimulatory molecule on T-cells (such as, for example, CD28 and/or 4-1BB). For example, in one aspect disclosed herein are methods for expanding an immune cell isolated from a subject for autologous immune therapy, comprising a) providing an immune and/or magnetic bead; wherein the beads comprises Protein L on its surface, wherein the bead comprise on its surface one or more single chain variable fragment (scFv) antibodies that bind a T cell inhibitory molecule, or a combination thereof, wherein the bead contains on its surface: one or more scFv that selectively bind CD3 and one or more scFv or ligands that bind CD28 and/or 4-1BB (such as, for example an anti-CD38 scFv and/or 4-BBL); and b) contacting the isolated immune cell with an effective amount of the beads to expand the immune cell in an amount effective for immunotherapy.

In one aspect, the methods of expanding immune cells and/or increasing the purity of immune cells can be used for expanding, enriching, and/or purifying CAR T cells, TILs, or MILs which can be used in immunotherapy. It is understood and herein contemplated that the use of said cells can comprise expanding CAR T cells, TILs, or MILs from a tissue from a subject. In one aspect, the CAR T cells, TILs, or MILs may be obtained from any tissue (such as, for example, biopsy, blood, urine, sputum, saliva, tissue lavage) in a subject by any means known in the art (tissue resection, biopsy phlebotomy, core biopsy). Because the tissue sample can be used, it can be advantageous to screen expanded CAR T cells, TILs, or MILs for desired activity (such as, for example, tumoricidal activity via expression of CD107). Thus, in one aspect, disclosed herein are methods for expanding tumor infiltrating lymphocytes for use in immunotherapy, comprising a) providing an immune and/or magnetic bead; wherein the beads comprises Protein L on its surface; b) expanding MILs and/or TILs from a biopsy of a tumor from a subject; c) screening the MILs and/or TILs for tumoricidal activity using flow cytometry to detect CD107 expression; and d) contacting the tumoricidal MILs and/or TILs with an effective amount of the beads to expand the tumoricidal MILs and/or TILs. In one aspect, the disclosed methods can further comprise infusing the expanded tumoricidal MILs and/or TILs into the subject in an effective amount to treat the tumor. In one aspect, the disclosed methods can further comprise beads wherein the beads comprise one or more single chain variable fragments (scFv) that bind a T cell inhibitory molecule, or a combination thereof, wherein the bead comprises on its surface one or more scFv that selectively bind CD3 and one or more scFv or ligand that binds a co-stimulatory molecule on T-cells.

It is understood and herein contemplated that the expansion of immune cells and/or increasing the purity of immune cells (such as, for example T cells, NK cells, or B cells) including CAR T cells, TILs, and MILs can occur ex vivo, in vitro, or in situ with the expansion occurring outside the subject and administration occurring after expansion. However, it is understood and herein contemplated that the expansion of immune cells including CAR T cells, TILs, and MILs can also occur in vivo by directly administering beads comprising an scFv that binds to a T cell inhibitory molecule, and an scFv recognizing an immune cell receptor (such as, for example) CD3 and scFv or ligands binding to co-stimulatory molecules (such as, CD28 and 4-1BB) directly to the subject in need of treatment.

In one aspect, the beads can further comprise on its surface a scFv or ligand that specifically binds a cytokine such as, IL2R, IL7R, IL12R, IL15R, IL18R, IL10R, or any combination thereof.

It is understood and herein contemplated that the disclosed methods can result in an expanded immune cell. Accordingly, in one aspect disclosed herein are immune cells produced by any method for expanding immune cells disclosed herein. In one aspect the methods of producing an immune cell (such as for example a CAR T cell) including expanded immune cells; wherein the method further comprises adding T cells to culture containers pre-treated with RetroNectin® and incubated with viral particles for gene transfer. The cells, beads and virus are incubated and the gene-modified cells are expanded.

A number of embodiments of the invention have been described. Nevertheless, it will be understood that various modifications may be made without departing from the spirit and scope of the invention. Accordingly, other embodiments are within the scope of the following claims.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A method for expanding an immune cell isolated from a subject for use in immune therapy, comprising
a) providing an immune and/or magnetic bead; wherein the beads comprises Protein L on its surface; and
b) contacting the isolated immune cell with an effective amount of the beads to expand the immune cell in an amount effective for immunotherapy.

2. The method of claim 1, wherein the immune cell comprises a chimeric antigen receptor (CAR) T cell, tumor infiltrating lymphocyte (TIL), or marrow-infiltrating lymphocyte (MIL).

3. The method of claim 1, wherein the immune cell comprises a natural killer (NK) cell, an NK-T cell, a cytokine-induced memory NK cell, a cytokine-induced killer (CIK) cell, or a γδ T cell.

4. The method of claim 1, wherein the beads further comprises one or more single chain variable fragment (scFv) antibodies that bind a T cell inhibitory molecule, or a combination thereof.

5. The method of claim 1, wherein the T cell inhibitory molecule comprises PD1, PDL1, or a combination thereof.

6. The method of claim 1, wherein the T cell inhibitory molecule comprises CTLA4, LAG3, TIM3, BTLA, CD160, 2B4, A2aR, KIR, or any combination thereof.

7. The method of claim 1, wherein the bead comprises on its surface one or more scFv or ligands that bind a co-stimulatory molecule on T-cells, one or more scFv that selectively bind CD3, or a combination thereof.

8. The method of claim 1, wherein the co-stimulatory molecule comprises CD28.

9. The method of claim 1, wherein the co-stimulatory molecule comprises 4-1BB.

10. An immune cell produced by the method of claim 1.

11. A method for increasing the purity of CAR T cells in a population of immune cells, comprising
a) providing an immune and/or magnetic bead; wherein the beads comprises Protein L on its surface; and
b) incubating the immune cell population with the beads for at least 8 hours; wherein the beads induces the CAR T cells to proliferate.

12. The method of claim 11, wherein the immune cell population comprises a chimeric antigen receptor (CAR) T cell and one or more of the T cell population comprising naïve T cells, activated T cells, memory T cells, γδ T cells, natural killer (NK) cell, an NK-T cell, a cytokine-induced memory NK cell, a cytokine-induced killer (CIK) cell, tumor infiltrating lymphocyte (TIL), and marrow-infiltrating lymphocyte (MIL).

13. The method of claim 11, wherein the immune cells are incubated with the beads for at least 9, 10, 11, 12, 13, 14, 15, 16, 17, 18, 19, 20, 21, 22, 23, 24, 26, 28, 30, 32, 34, 36, or 48 hours.

14. The method of claim 11, wherein the beads further comprises one or more single chain variable fragment (scFv) antibodies that bind a T cell inhibitory molecule, or a combination thereof.

15. The method of claim 11, wherein the T cell inhibitory molecule comprises PD1, PDL1, or a combination thereof.

16. The method of claim 11, wherein the T cell inhibitory molecule comprises CTLA4, LAG3, TIM3, BTLA, CD160, 2B4, A2aR, KIR, or any combination thereof.

17. The method of claim 11, wherein the bead comprises on its surface one or more scFv or ligands that bind a co-stimulatory molecule on T-cells, one or more scFv that selectively bind CD3, or a combination thereof.

18. The method of claim 11, wherein the co-stimulatory molecule comprises CD28.

19. The method of claim 11, wherein the co-stimulatory molecule comprises 4-1BB.

20. The method of claim 11, wherein the immune cell population consists essentially of a T cell population.
